(19) United States
(12) Patent Application Publication
(10) Pub. No.: US 2011/0250161 A1
(43) Pub. Date: Oct. 13, 2011

(54) VEGETABLE BASED THREE COMPONENT HAIR RELAXER

(75) Inventors: David Jarvis, Jacksonville, FL (US); Yogini Panchal, Orange Park, FL (US); Kelly R. Simpson, Jacksonville, FL (US); Carl A. Helmers, Jacksonville, FL (US)

(73) Assignee: ROUX LABORATORIES, INC. D/B/A COLOMER USA, Jacksonville, FL (US)

(21) Appl. No.: 12/758,279

(22) Filed: Apr. 12, 2010

(51) Int. Cl. A61K 8/97; A61Q 5/04

(52) U.S. Cl. 424/70.2

(57) ABSTRACT

The disclosure relates to a three part, vegetable-based, no-lye composition for relaxing human hair that excludes petroleum derived raw materials. The disclosure relates to a cream base, a hydroxide cream dispersion, and an aqueous activator solution. The disclosure further relates to methods for relaxing human hair with minimal damage to the hair and improved performance compared to existing no-lye relaxer products.

### FIELD OF THE INVENTION

The disclosure relates to a three part, vegetable-based, no-lye composition for relaxing human hair that excludes petroleum derived raw materials. The disclosure relates to a cream base, a hydroxide cream dispersion, and an aqueous activator solution. The disclosure further relates to methods for relaxing human hair with minimal damage to the hair and improved performance compared to existing no-lye relaxer products.

### BACKGROUND OF THE INVENTION

Relaxers are chemical solutions used for the purpose of straightening, softening or loosening curled or wavy hair. Generally these chemical compositions contain soluble alkaline earth materials such as sodium or calcium hydroxide. Organic bases such as guanidine hydroxide are also employed as relaxers.

Sodium hydroxide, used in products referred to as “lye” relaxers, provides the longest lasting and most dramatic effects. However, because of their high alkalinity (i.e., a pH of about 14), solutions of sodium hydroxide have a pronounced tendency to damage skin and hair. For this reason, alternatives to lye products have been developed and marketed. Chief among these alternatives are relaxer products referred to as “no-lye” relaxers.

A no-lye relaxer is disclosed in co-pending application Ser. No. 12/176,285, the entire contents of which is hereby incorporated by reference for all purposes.

“No-lye” relaxer systems have a lower pH than lye relaxers but are still extremely alkaline, usually having a pH of about 13, and have the potential to weaken the hair making it extremely susceptible to breaking and further damage. For this reason, a pre-protective petroleum “base cream” is frequently applied to help protect the scalp against chemical irritation. After the hair has been chemically relaxed, a neutralizing formula may be applied to lower the pH and reduce the potential for hair breakage. Frequently, a conditioner is applied to the hair to restore some of the natural oils and proteins removed by the relaxer chemicals.

“Lye” relaxers utilize the inorganic base sodium hydroxide to liberate hydroxide ions (OH—) necessary for the high pH lanthionization of keratin and the resultant straightening of the hair fiber.

“No-lye” relaxers utilize the organic chemical base, guanidine, typically present in the form of guanidine hydroxide, as the straightening agent that produces hydroxide ions (OH—) necessary for keratin reduction. No-lye relaxers are typically supplied as two component systems. A first component contains a hydroxide reagent, typically calcium hydroxide. The reagent of the second component is typically guanidine carbonate. On mixing, the two reagents undergo a substitution reaction to produce soluble guanidine hydroxide.

The first component, is typically a water-in-oil (W/O) cream emulsion containing calcium hydroxide as the reagent at 3-8% by weight. This first component is typically referred to as the “base” or “relaxer base.”

The second component is a liquid “activator,” a water solution of a salt of a relatively strong organic base with an anion capable of being precipitated by an alkaline earth metal ion under alkaline conditions. Typically, the activator is comprised of an aqueous solution of guanidine carbonate as the limiting reagent. The guanidine carbonate is typically maintained at or near its saturation point in water, approximately 29.5 weight percent (wt %). Upon mixing, the guanidine carbonate component of the activator solution reacts with the base, e.g. calcium hydroxide, forming guanidine hydroxide and precipitated calcium carbonate.

Guanidine hydroxide is not stable for prolonged periods of time and decomposes into ammonia and urea within 6-12 hours after mixing. The guanidine hydroxide elevates the pH of the product to about 13 and thereby allows for successful relaxation of the hair. Products of this type normally do not require a pre-protective application of petrolatum or similar composition. These products are typically called “no-lye products.”

The high pH relaxer compositions available today utilize very much the same chemistry in that they all rely on the generation of hydroxide to relax the hair via the substitution reaction described above. They also rely on the presence of petroleum distillates such as mineral oil and petrolatum in the cream base formulation to provide skin and hair protection during the relaxation process. The high pH of these formulations presents challenges to the formulator. Modern compositions contain skin protectants such as mineral oil and petrolatum and also waxes, conditioning agents, and humectants. The high pH of the relaxer cream base prevents the inclusion of many ingredients that would have greater beneficial effect for the consumer.

The typical relaxer base containing calcium hydroxide has a pH in the range of 11-12.5 before activation with guanidine carbonate. After activation, this pH will increase 15 fold or more. These high pH values present difficulties because at pH above 8 many beneficial organic ingredients are destroyed. At this elevated pH only the most basic and chemically resistant materials survive for any length of time. While many modern products may include some organic materials in their relaxer creams or bases, the longevity and stability of these materials is very highly suspect at the pH mentioned above. As a result, many relaxers deliver poor performance and may irritate the skin if not used specifically as directed.

Petroleum distillates, such as mineral oil and petrolatum, are chemically stable at high pH, typically 11-12.5, generated by the presence of either calcium hydroxide or guanidine carbonate. Therefore, they are ubiquitously present in hair relaxer preparations. Mineral oil and petrolatum are the only ingredients which are both stable at high pH and are recognized in the art to provide sufficient lubricity, occlusivity, and emolliency to protect the skin during the relaxation process. However, the modern consumer increasingly demands “green” products such as those derived from renewable resources and not containing materials derived from petroleum.

Prior art no-lye relaxers comprise a hydroxide containing component and a guanidine carbonate component, each of which is very alkaline having a ph above 10. Because of consumers desire for “green” products, there exists a need to provide “petroleum-free” hair-care ingredients. However, “petroleum-free” hair-care ingredients tend to be alkali-sensitive and tend to break-down when stored at the high-pH conditions of the relaxer base and guanidine activator. There exists a need for a lower-pH cream component to carry “petroleum-free,” alkali-sensitive conditioning components.

### SUMMARY OF THE INVENTION

The present disclosure addresses unfelt needs in the art by providing a “green,” petroleum-free product. The present invention further addresses unmet needs by providing a lower-pH cream component to carry “petroleum-free,” alkali-sensitive conditioning components.

An aspect of the disclosure relates to a petroleum-free, vegetable-based, no-lye hair relaxer composition. According to aspects of the disclosure, there is provided a petroleum-free, cream-base having a pH of from about 4 to about 10. According to aspects of the disclosure, there is provided a petroleum-free, hydroxide cream dispersion. According to aspects of the disclosure, there is provided a petroleum-free, guanidine carbonate solution.

According to aspects of the disclosure, the cream-base comprises a water emulsion of one or more oleaginous components. According to aspects of the disclosure, non-limiting examples of suitable oleaginous components may include: non-ionic emulsifiers, anionic emulsifiers, cationic conditioners, amphoteric emulsifiers, zwitterionic vegetable-based oils, vegetable-based waxes, lubricants, and mixtures thereof.

According to aspects of the disclosure, the cream-base may comprise sugars such as, but not limited to fructose, glucose, maltose, saccharose and natural combinations of these such as honey.

According to aspects of the disclosure, the cream-base may comprise herbs such as Apocynum Cannabinuin Root, Urtica Dioica, Rosmarinus Officinalis Leaf, Arctium Lappa Root, Betula Alba, Rosa Canina Fruit, Chondrus Crispus, Tussilago Farfara Flower, Prunus Serotina Bark, Taraxacum Officinale, Sambucus Nigra, Equissetum Hiemale, Echinacea Purpurea and mixtures thereof.

According to aspects of the disclosure, the cream-base may comprise an oil in water emulsion.

According to aspects of the disclosure, the cream-base has a pH of from about 4 to about 10.

According to aspects of the disclosure, the vegetable-based oil comprises at least one natural oil which may include, but is not limited to, aloe, corn, sunflower, olive, jojoba, safflower, peanut, coconut, hydrogenated soybean oil, hydrogenated cottonseed oil, Shea oil, palm oil, grapeseed oil, argan oil, corn-derived propanediol, and mixtures thereof.

According to an aspect, the cream-base may comprise esters, such as glyceryl stearate; fatty acids, from coconut, soybean, olive and other biological oils; proteins and peptides; herbs and plants and biologicals extracted from herbs and plants.

According to aspects of the disclosure, there is provided a hydroxide cream dispersion. According to aspects of the disclosure, the hydroxide cream dispersion may comprise an alkaline material selected from the group consisting of alkali metal hydroxides, alkaline earth metal hydroxides, and mixtures thereof. According to an aspect of the disclosure, the concentration of the hydroxide compound in the dispersion may vary from about 19 weight percent (wt %) to about 40 wt %.

According to an aspect of the disclosure, the hydroxide cream dispersion may comprise an alkaline material selected from the group consisting of C₂-C₁₀-primary amines, C₂-C₁₀-secondary amines, C₂-C₁₀-tertiary amines, and mixtures thereof.

According to an aspect of the disclosure, the hydroxide cream dispersion may further comprise naturally-derived surfactants, glycol solvents, pearling agents, suspending agents, and mixtures thereof.

According to an aspect of the disclosure, the hydroxide cream dispersion may further comprise plant-derived propanediol. The plant, or other biomass-derived propanediol may be corn-derived propanediol. Some or all of the propanediol may be substituted by biomass-derived glycerin.

According to aspects of the disclosure, there is provided an “activator.” According to aspects of the disclosure, the activator is a water solution of a salt of a relatively strong organic base with an anion capable of being precipitated by an alkaline earth metal ion under alkaline conditions. Typically, the activator is comprised of an aqueous solution of guanidine carbonate as the limiting reagent. The guanidine carbonate is typically maintained at or near its saturation point in water, approximately 29.5 wt %. Upon mixing, the guanidine carbonate component of the activator solution reacts with the base, e.g. calcium hydroxide, forming guanidine hydroxide and precipitated calcium carbonate.

According to aspects of the disclosure, there is provided a guanidine carbonate solution. According to an aspect of the disclosure, the guanidine carbonate solution comprises guanidine carbonate at a concentration of from about 25 wt % to about 35 wt %.

According to an aspect of the disclosure, guanidine carbonate is a non-limiting example of an activator, an anion salt of a strong base suitable for purposes of the invention. A non-limiting, suitable strong base may be selected from the group consisting of guanidine, N-methyl guanidine, symmetrical dimethylaminoguanidine, asymmetrical dimethylaminoguanidine, acetamidine, dimethylaminoamidine, aminoamidine, acetamide, and mixtures thereof.

According to an aspect of the disclosure, non-limiting, suitable anions may be selected from the group consisting of carbonate, sulfate, sulfite, fluoride, oxalate, tartrate, laurate, alginate, and mixtures thereof.

According to an aspect of the disclosure, the activator solution may further comprise xanthan and/or cellulose gum.

According to aspects of the disclosure, there are provided methods of relaxing human hair. According to an aspect of the disclosure, the methods may comprise providing a cream-base emulsion, providing a hydroxide cream dispersion, and providing an aqueous guanidine carbonate solution, mixing the cream-base with the hydroxide dispersion and with the guanidine carbonate solution to form an aqueous mixture having a pH of at least 11.

According to an aspect of the disclosure, the methods may provide a petroleum-free cream-base emulsion, a petroleum-free, hydroxide cream dispersion, and a petroleum-free, aqueous guanidine carbonate solution.

According to an aspect of the disclosure, the methods may further comprise applying the aqueous mixture to human hair for a period of time sufficient to effect a desired amount of relaxation and removing said aqueous mixture from the hair with a rinsing liquid.

According to an aspect of the disclosure, a method is provided wherein a guanidine hydroxide is formed by an in situ reaction between the guanidine of the guanidine salt solution and the alkali metal and/or the alkaline earth metal hydroxide of the hydroxide cream dispersion.

According to an aspect of the disclosure, a method is provided wherein a hydroxide of a strong base, such as guanidine hydroxide is formed by an in situ reaction between the guanidine carbonate of the activator solution and calcium hydroxide of the hydroxide cream dispersion.

Still other aspects and advantages of the present invention will become readily apparent by those skilled in the art from the following detailed description, wherein it is shown and described preferred embodiments of the invention, simply by way of illustration of the best mode contemplated of carrying out the invention. As will be realized the invention is capable of other and different embodiments, and its several details are capable of modifications in various obvious respects, without departing from the invention. Accordingly, the description is to be regarded as illustrative in nature and not as restrictive.

### BRIEF DESCRIPTION OF DRAWINGS

FIG. 1 compares the tensile strength of hair fibers treated by compositions of the present invention to hair fibers treated by conventional compositions.

### DETAILED DESCRIPTION OF AN EMBODIMENT

Modern consumers increasingly demand “green” products such as those derived from renewable resources, e.g., biomass, and not containing materials derived from petroleum. An aspect of the disclosure provides “petroleum-free” materials that are recognized in the art to provide sufficient lubricity, occlusivity, and emolliency to protect the skin during the relaxation process.

The term “petroleum-free” means materials that are not refined from petroleum or are petroleum distillates. The term petroleum-free excludes chemicals synthesized from petroleum distillates. The term petroleum-free is meant to encompass compounds derived from biological sources. A non-limiting example of a petroleum-free compound is propanediol derived from corn.

Ethoxylated lauryl alcohol (e.g., laureth-23) is an example of a compound that is derived in part from biological sources and in part from petrochemicals. Laureth-23 is formed by reacting (ethoxylating) lauryl alcohol, a fatty alcohol derived from coconut, with petroleum-derived ethylene oxide. For purposes of the present disclosure, fatty alcohol ethoxylates are considered to be “petroleum-free,” biologically-based compounds.

Polysorbates, such as Polysorbate-60, are surfactants produced by reacting the naturally occurring sugar-like alcohol, sorbitol, with ethylene oxide to form a polyethoxylated sorbitan which is then reacted with fatty acids obtained from vegetable fats and oils such as, but not limited to: lauric, palmitic, stearic, and oleic acids. These compounds share substantial similarities with fatty alcohol ethoxylates. For purposes of the present disclosure, polysorbates are considered to be “petroleum-free,” biologically-based compounds.

Pegylated lanolins, such as PEG-75 lanolin, are formed by ethoxylating lanolin, a secretory product of sheep sebaceous glands obtained from sheared wool, with ethylene oxide to form polyethylene glycol lanolin. For purposes of the present disclosure, pegylated lanolins are considered to be “petroleum-free,” biologically-based compounds.

Alkyldimonium hydroxypropyl-derived polypeptides and proteins, such as cocodimonium hydroxypropyltrimonium hydrolyzed keratin, are proteins where a derivatized hydroxypropyl group is covalently-bound to a lysine side-chain amino group. The hydroxypropyl is derived with an alkyl group usually derived from coconut or other biological oils. For purposes of the present disclosure, alkyldimonium hydroxypropyl-derived polypeptides and proteins are considered to be “petroleum-free,” biologically-based compounds.

For purposes of the present disclosure, cationic guar gums, such as guar hydroxypropyltrimonium chloride (known in the trade as Jaguar C-13S) are considered to be “petroleum-free,” biologically-based compounds. Guar gum is obtained from the endosperm of Cyamopsis tetragonolobus and is made up of straight chain mannan groupings with relatively regular branching on every second mannose by a single galactose unit. Cationic guar gums are obtained by reacting reactive quaternary ammonium compounds with hydroxyl groups on the galactose sugars of guar to obtain the corresponding cationic derivative.

For purposes of the present disclosure, the term “petroleum-free” includes any compound that is typically used as a cosmetic ingredient that is substantially derived from a biological source and that may be esterified or otherwise chemically derived.

The term “vegetable-based” means compounds such as, but not limited to, oils, waxes, and other oleaginous materials derived from a biological such as, but not limited to corn, soy, and coconut.

According to aspects of the disclosure, there is provided an “activator.” According to aspects of the disclosure, the activator is a water solution of a salt of a relatively strong organic base with an anion capable of being precipitated by an alkaline earth metal ion under alkaline conditions. Typically, the activator is comprised of an aqueous solution of guanidine carbonate as the limiting reagent. The guanidine carbonate is typically maintained at or near its saturation point in water, approximately 29.5 wt %. Upon mixing, the guanidine carbonate component of the activator solution reacts with the base, e.g. calcium hydroxide, forming guanidine hydroxide and precipitated calcium carbonate. The pH of the mixture is thus elevated through the reaction shown in Scheme I.

Guanidine carbonate+calcium hydroxide→Guanidine hydroxide+calcium carbonate [Scheme I]

The reaction occurs rapidly at room temperature with elevation of pH to a range of 13-14.

The present disclosure relates to a three-component, vegetable-based, no-lye hair relaxer.

A first component is a petroleum-free, cream-base having a pH of from about 4 to about 10. The cream-base comprises an oil-in-water (O/W) emulsion of one or more oleaginous components selected from the group consisting of non-ionic emulsifiers, anionic emulsifiers, cationic conditioners, amphoteric emulsifiers, zwitterionic vegetable-based oils, vegetable-based waxes, lubricants, and mixtures thereof. Any cosmetically-acceptable oleaginous component may be suitable, provided that it originates from a non-petroleum based source. Persons of skill in the art may consult standard trade references such as the International Cosmetic Ingredient Dictionary and Handbook, published by the Cosmetic, Toiletry, and Fragrance Association.

The cream base may further comprise sugars such as fructose, glucose, maltose, saccharose and natural combinations of these such as honey.

The cream base may further comprise herbs such as Apocynum Cannabinum Root, Urtica Dioica, Rosmarinus Officinalis Leaf, Arctium Lappa Root, Betula Alba, Rosa Canina Fruit, Chondrus Crispus, Tussilago Farfara Flower, Prunus Serotina Bark, Taraxacum Officinale, Sambucus Nigra, Equissetum Hiemale, Echinacea Purpurea and mixtures thereof.

The disclosure relates to a cream-base having a pH of from about 4 to about 10.

For purposes of the cream-base, the vegetable-based oil may comprise at least one natural oil selected from the group consisting of aloe, corn, sunflower, olive, jojoba, safflower, peanut, coconut, hydrogenated soybean oil, hydrogenated cottonseed oil, corn-derived propanediol, Shea oil, palm oil, grapeseed oil, argan oil, and mixtures thereof.

A second component may be a petroleum-free, hydroxide cream dispersion. The hydroxide cream dispersion comprises an alkaline material selected from the group consisting of alkali metal hydroxides, alkaline earth metal hydroxides, C₂-C₁₀-primary amines, C₂-C₁₀-secondary amines, C₂-C₁₀-tertiary amines, and mixtures thereof.

Suitable alkaline materials include those alkaline earth oxides which react in water solution to produce hydroxides.

The hydroxide cream dispersion may further comprise naturally-derived, i.e. non-petroleum based, surfactants, glycol solvents, pearling agents, suspending agents, and mixtures thereof.

According to an aspect of the disclosure, the hydroxide cream dispersion further comprises plant-derived propanediol. The plant, or other biomass-derived propanediol may be corn-derived propanediol. Some or all of the propanediol may be substituted by biomass-derived glycerin.

The disclosure further relates to a petroleum-free, activator solution. The activator is a water solution of a salt of a relatively strong organic base with an anion capable of being precipitated by an alkaline earth metal ion under alkaline conditions. A preferred water-soluble salt of a strong organic base is usually guanidine carbonate solution.

Other strong organic bases which may be substituted for some or all of the guanidine, include, but are not limited to: N-methyl guanidine, symmetrical and asymmetrical dimethylaminoguanidine, acetamidine, dimethylaminoamidine, aminoamidine and acetamide.

The anion capable of being precipitated by an alkaline earth metal ion under alkaline conditions may include carbonate, sulfate, sulfite, fluoride, oxalate, tartrate, laurate, and alginate anions.

According to an aspect of the disclosure, the activator solution further comprises xanthan and/or cellulose gum.

Typically, the hydroxide base is calcium hydroxide. Typically, the activator is guanidine carbonate. Upon mixing in aqueous solution, a reaction ensues resulting in the formation of guanidine hydroxide and precipitated calcium carbonate. Typically, the hydroxide base has a pH in the range of 11 to 12.5. Typically, formation of guanidine hydroxide results in an elevation of the pH by about 15-fold.

The present disclosure further relates to methods of relaxing human hair. The present methods include providing a petroleum-free, cream-base emulsion, providing a petroleum-free, hydroxide cream dispersion, and providing a petroleum-free, aqueous activator solution. The methods include mixing the cream-base with said hydroxide dispersion and the activator solution to form an aqueous mixture having a pH of at least 11. Preferably, the pH is at least 12; and more preferably, the pH is at least 13. The methods include applying the aqueous mixture to human hair for a period of time sufficient to affect a desired amount of relaxation; and removing the aqueous mixture from the hair with a rinsing liquid.

### Example

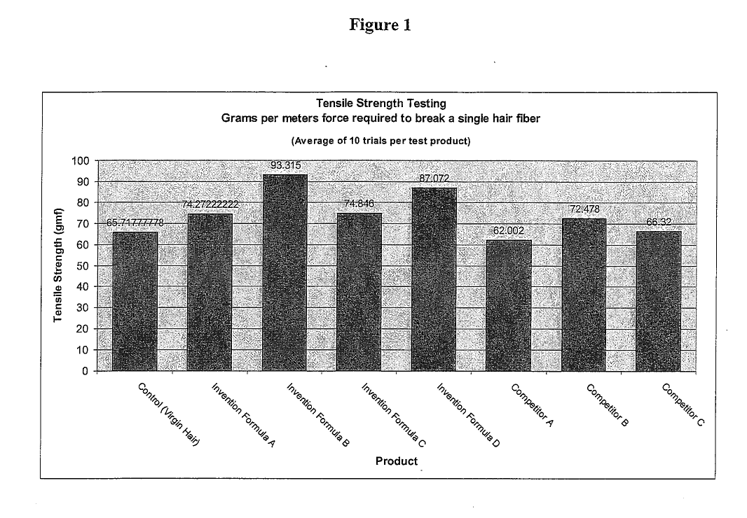

FIG. 1 presents the results of relaxing trials performed with compositions of the present invention compared against prior art compositions. Each bar represents the average of ten trials. Control (untreated hair) withstood an average force of 65.72 grams per meter before breaking. Hair fibers treated with compositions of the present invention were stronger than control fibers. Hair treated with a basic composition of the present invention (A) withstood an average force of 74.3 grams per meter before breaking. Compositions of the present invention were also supplemented with either 0.14 wt % herbs (B), 1.0 wt % honey (C), or 1.5 wt % cocodimonium hydroxypropyl hydrolyzed keratin (D). Hair fibers treated with supplemented solutions of the present invention were stronger that those treated with a un-supplemented composition of the present invention. In contrast, identical treatment with competitive compositions left hair fibers weaker than untreated fibers.

1. A vegetable-based, no-lye hair relaxer comprising:
a cream-base having a pH of from about 4 to about 10;
a hydroxide cream dispersion; and,
a activator solution.

2. The vegetable-based, no-lye hair relaxer of claim 1, wherein said relaxer is petroleum-free.

3. The vegetable-based, no-lye hair relaxer of claim 1, wherein said cream-base comprises a water emulsion of one or more oleaginous components selected from the group consisting of non-ionic emulsifiers, anionic emulsifiers, amphoteric emulsifiers, cationic conditioners, zwitterionic vegetable-based oils, vegetable-based waxes, lubricants, and mixtures thereof.

4. The vegetable-based, no-lye hair relaxer of claim 1, wherein said cream-base further comprises a sugar selected from the group consisting of fructose, glucose, maltose, saccharose, natural combinations thereof, and mixtures thereof.

5. The vegetable-based, no-lye hair relaxer of claim 1, wherein said cream-base further comprises an herb selected from the group consisting of Apocynum Cannabinum Root, Urtica Dioica, Rosmarinus Officinalis Leaf, Arctium Lappa Root, Betula Alba, Rosa Canina Fruit, Chondrus Crispus, Tussilago Farfara Flower, Prunus Serotina Bark, Taraxacum Officinale, Sambucus Nigra, Equissetum Hiemale, Echinacea Purpurea, and mixtures thereof.

6. The vegetable-based, no-lye hair relaxer of claim 1, wherein said cream-base has a pH of from about 4 to about 10.

7. The vegetable-based, no-lye hair relaxer of claim 3, wherein said vegetable-based oil comprises at least one natural oil selected from the group consisting of aloe, corn, sunflower, olive, jojoba, safflower, peanut, coconut, hydrogenated soybean oil, hydrogenated cottonseed oil, corn-derived propanediol, Shea oil, palm oil, grapeseed oil, argan oil, and mixtures thereof.

8. The vegetable-based, no-lye hair relaxer of claim 1, wherein said hydroxide cream dispersion comprises an alkaline material selected from the group consisting of alkali metal hydroxides, alkaline earth metal hydroxides, C₂-C₁₀-primary amines, C₂-C₁₀-secondary amines, C₂-C₁₀-tertiary amines, and mixtures thereof.

9. The vegetable-based, no-lye hair relaxer of claim 8, wherein said hydroxide cream dispersion further comprises naturally-derived surfactants, glycol solvents, pearling agents, suspending agents, and mixtures thereof.

10. The vegetable-based, no-lye hair relaxer of claim 1, wherein said hydroxide cream dispersion further comprises a biomass-derived agent selected from the group consisting of propanediol, glycerin, xanthan gum, cellulose gum, and mixtures thereof.

11. The vegetable-based, no-lye hair relaxer of claim 1, wherein said activator solution comprises a strong base selected from the group consisting of guanidine, N-methyl guanidine, symmetrical dimethylaminoguanidine, asymmetrical dimethylaminoguanidine, acetamidine, dimethylaminoamidine, aminoamidine, acetamide, and mixtures thereof.

12. The vegetable-based, no-lye hair relaxer of claim 11, wherein said activator solution comprises an anion selected from the group consisting of carbonate, sulfate, sulfite, fluoride, oxalate, tartrate, laurate, alginate, and mixtures thereof.

13. A method of relaxing human hair comprising:
providing a cream-base emulsion;
providing a hydroxide cream dispersion;
providing an aqueous activator solution;
mixing said cream-base with said hydroxide dispersion and said activator solution to form an aqueous mixture having a pH of at least 11;
applying said aqueous mixture to human hair for a period of time sufficient to effect a desired amount of relaxation; and
removing said aqueous mixture from the hair with a rinsing liquid.

14. The method of relaxing human hair according to claim 13, wherein each of said cream base, hydroxide cream, and activator is petroleum-free.

15. The method of relaxing human hair according to claim 13, wherein said cream-base comprises a water emulsion of one or more oleaginous components selected from the group consisting of non-ionic emulsifiers, anionic emulsifiers, amphoteric emulsifiers, cationic conditioners, zwitterionic vegetable-based oils, vegetable-based waxes, lubricants, and mixtures thereof.

16. The method of relaxing human hair according to claim 15, wherein said cream-base further comprises a sugar selected from the group consisting of fructose, glucose, maltose, saccharose, natural combinations thereof, and mixtures thereof.

17. The method of relaxing human hair according to claim 15, wherein said cream-base further comprises an herb selected from the group consisting of Apocynum Cannabinum Root, Urtica Dioica, Rosmarinus Officinalis Leaf, Arctium Lappa Root, Betula Alba, Rosa Canina Fruit, Chondrus Crispus, Tussilago Farfara Flower, Prunus Serotina Bark, Taraxacum Officinale, Sambucus Nigra, Equissetum Hiemale, Echinacea Purpurea, and mixtures thereof.

18. The method of relaxing human hair according to claim 15, wherein said cream-base has a pH of from about 4 to about 10.

19. The method of relaxing human hair according to claim 15, wherein said cream-base further comprises wherein said vegetable-based oil comprises at least one natural oil selected from the group consisting of aloe, corn, sunflower, olive, jojoba, safflower, peanut, coconut, hydrogenated soybean oil, hydrogenated cottonseed oil, Shea oil, palm oil, grapeseed oil, argan oil, biomass-derived propanediol, and mixtures thereof.

20. The method of relaxing human hair according to claim 13, wherein said hydroxide cream dispersion comprises an alkaline material selected from the group consisting of alkali metal hydroxides, alkaline earth metal hydroxides, C₂-C₁₀-primary amines, C₂-C₁₀-secondary amines, C₂-C₁₀-tertiary amines, and mixtures thereof.

21. The method of relaxing human hair according to claim 20, wherein said hydroxide cream dispersion further comprises naturally-derived surfactants, glycol solvents, pearling agents, suspending agents, and mixtures thereof.

22. The method of relaxing human hair according to claim 20, wherein said hydroxide cream dispersion further comprises a biomass-derived agent selected from the group consisting of propanediol, glycerin, xanthan gum, and cellulose gum.

23. The method of relaxing human hair according to claim 13, wherein said activator solution comprises a strong base selected from the group consisting of guanidine, N-methyl guanidine, symmetrical dimethylaminoguanidine, asymmetrical dimethylaminoguanidine, acetamidine, dimethylaminoamidine, aminoamidine, acetamide, and mixtures thereof.

24. The method of relaxing human hair according to claim 23, wherein said activator solution comprises an anion selected from the group consisting of carbonate, sulfate, sulfite, fluoride, oxalate, tartrate, laurate, alginate, and mixtures thereof.

25. The method of relaxing human hair according to claim 13, wherein a hydroxide of a strong base is formed by an in situ reaction between said activator solution and said hydroxide cream dispersion.
